(19) United States
(12) Patent Application Publication
(10) Pub. No.: US 2022/0251254 A1
(43) Pub. Date: Aug. 11, 2022

(54) MALEIMIDE, CURABLE RESIN COMPOSITION, AND CURED PRODUCT

(71) Applicant: DIC Corporation, Tokyo (JP)

(72) Inventors: Tomohiro Shimono, Ichihara-shi (JP); Tatsuya Okamoto, Ichihara-shi (JP)

(73) Assignee: DIC Corporation, Tokyo (JP)

(21) Appl. No.: 17/622,779

(22) PCT Filed: Feb. 20, 2020
(86) PCT No.: PCT/JP2020/006820

§ 371 Date: Dec. 24, 2021

(30) Foreign Application Priority Data
Apr. 26, 2019 (JP) 2019-086451

(51) Int. Cl. C08F 22/40

(52) U.S. Cl. CPC C08F 22/40

(57) ABSTRACT

To provide a curable resin composition of which a cured product has excellent heat-resistance and dielectric properties, and a cured product thereof, and a prepreg, a circuit board, a build-up film, a semiconductor sealing material, and a semiconductor apparatus having both excellent heat resistance and dielectric properties. The invention has an indane scaffold represented by the following general formula (1):

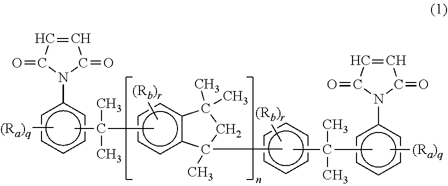

in formula (1), Ra's are each independently an alkyl group, an alkyloxy group or an alkylthio group, which has 1 to 10 carbon atoms, an aryl group, an aryloxy group or an arylthio group, which has 6 to 10 carbon atoms, a cycloalkyl group having 3 to 10 carbon atoms, a halogen atom, a nitro group, a hydroxyl group, or a mercapto group, and q represents an integer value of 0 to 4.

### TECHNICAL FIELD

The present invention relates to a maleimide having an indane scaffold, a curable resin composition containing the maleimide, and a cured product obtained by the curable resin composition.

### BACKGROUND ART

As a material for a circuit board for an electronic device, a prepreg obtained by impregnating a glass cloth with a thermosetting resin such as an epoxy resin and a BT (bismaleimide-triazine) resin and heat-drying, a laminated board obtained by heat-curing the prepreg, and a multilayer board obtained by combining and heat-curing the laminated board and the prepreg are widely used. In particular, semiconductor package substrates are becoming thinner, and warpage of the package substrate during mounting becomes a problem. Therefore, in order to suppress the warpage, a material exhibiting high heat resistance is required.

Further, in recent years, the speed and frequency of signals have been increased, and it has been desired to provide a thermosetting resin composition that provides a cured product exhibiting a sufficiently low dielectric loss tangent while maintaining a sufficiently low dielectric constant in these environments.

In particular, recently, further improvement of performance represented by heat resistance and dielectric properties, and materials and compositions having both properties have been required in various electrical material applications, especially in advanced material applications.

In response to these demands, a maleimide resin is attracting attention as a material having heat resistance and low dielectric constant and low dielectric loss tangent. However, although the conventional maleimide resin exhibits high heat resistance, its dielectric constant and dielectric loss tangent values fail to reach the levels required for advanced material applications. In addition, the conventional maleimide resin is inferior in handleability due to its poor solvent solubility, and thus it is strongly desired to develop a resin that has excellent solvent solubility and exhibits further low dielectric constant and low dielectric loss tangent while maintaining heat resistance.

Moreover, a cured product using the conventional maleimide resin is inferior in brittleness resistance to an epoxy resin or the like, and an obtained cured product is also required to have elastic behavior and flexibility.

Under such circumstances, as a cyanate ester-based material having both high dielectric properties and heat resistance, a resin composition obtained by blending a phenol novolac type cyanate ester resin, a bisphenol A cyanate ester resin, and a non-halogen epoxy resin is known (see Patent Literature 1).

However, although the resin composition described in Patent Literature 1 has improved heat resistance and dielectric properties in the cured product to some extent, the heat resistance still has been below the level required in recent years.

### CITATION LIST

### Patent Literature

PTL 1: JP-A-2004-182850

### SUMMARY OF INVENTION

### Technical Problem

Therefore, a problem to be solved by the invention is to provide a cured product having excellent brittleness resistance, elastic behavior, flexibility, heat resistance, and dielectric properties by using a maleimide that is excellent in solvent solubility, fluidity during heat melting, and handleability.

### Solution to Problem

Therefore, as a result of diligent studies to solve the above problem, the inventors of the invention have found a maleimide having excellent solvent solubility, fluidity during heat melting, and handleability, and having an indane scaffold capable of contributing to brittleness resistance, heat resistance, low dielectric constant and low dielectric loss tangent, and that a cured product obtained by a curable resin composition containing the maleimide is excellent in brittleness resistance, elastic behavior, flexibility, heat resistance, and dielectric properties, and the inventors have completed the invention.

That is, the invention has an indane scaffold represented by the following general formula (1).

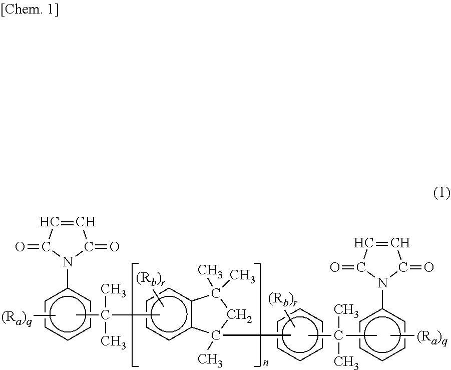

(In formula (1), Ra's are each independently an alkyl group, an alkyloxy group or an alkylthio group, which has 1 to 10 carbon atoms, an aryl group, an aryloxy group or an arylthio group, which has 6 to 10 carbon atoms, or a cycloalkyl group having 3 to 10 carbon atoms, a halogen atom, a nitro group, a hydroxyl group, or a mercapto group, and q represents an integer value of 0 to 4. When q is 2 to 4, Ra's may be the same or different from each other in a same ring. Rb's are each independently an alkyl group, an alkyloxy group or an alkylthio group, which has 1 to 10 carbon atoms, an aryl group, an aryloxy group or an arylthio group, which has 6 to 10 carbon atoms, a cycloalkyl group having 3 to 10 carbon atoms, a halogen atom, a hydroxyl group, or a mercapto group, and r represents an integer value of 0 to 3. When r is 2 to 3, Rb's may be the same or different from each other in a same ring. n is an average number of repeating units, and represents a numerical value of 0.95 to 10.0.)

The maleimide of the invention preferably has a molecular weight distribution (Mw/Mn) obtained by GPC measurement of 1.0 to 4.0.

In the maleimide of the invention, it is preferable that Ra in the formula (1) is at least one selected from the group consisting of an alkyl group having 1 to 4 carbon atoms, a cycloalkyl group having 3 to 6 carbon atoms, and an aryl group having 6 to 10 carbon atoms.

In the maleimide of the invention, it is preferable that q in the formula (1) is 2 to 3.

In the maleimide of the invention, it is preferable that r in the formula (1) is 0 and Rb is a hydrogen atom.

In the maleimide of the invention, it is preferable that r in the formula (1) is 1 to 3, and Rb is at least one selected from the group consisting of an alkyl group having 1 to 4 carbon atoms, a cycloalkyl group having 3 to 6 carbon atoms, and an aryl group having 6 to 10 carbon atoms.

The maleimide of the invention preferably contains 32 area % or less of maleimide in which n is 0 in 100% by mass of a total amount of the maleimide.

A curable resin composition of the invention preferably contains the maleimide.

A cured product of the invention is preferably obtained by the curing agent composition.

### Advantageous Effects of Invention

The maleimide of the invention has excellent solvent solubility, fluidity during heat melting, and handleability and is further capable of contributing to brittleness resistance, heat resistance, low dielectric constant and low dielectric loss tangent. Therefore, a cured product obtained by a curable resin composition containing the maleimide is excellent in brittleness resistance, elastic behavior, flexibility, heat resistance and dielectric properties, and is useful.

### DESCRIPTION OF EMBODIMENTS

The invention will be described in detail below.

The invention has an indane scaffold represented by the following general formula (1).

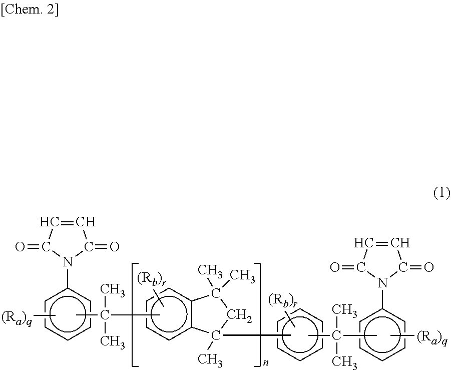

In the above general formula (1), Ra's are each independently an alkyl group, an alkyloxy group or an alkylthio group, which has 1 to 10 carbon atoms, an aryl group, an aryloxy group or an arylthio group, which has 6 to 10 carbon atoms, or a cycloalkyl group having 3 to 10 carbon atoms, a halogen atom, a nitro group, a hydroxyl group, or a mercapto group, and q represents an integer value of 0 to 4. When q is 2 to 4, Ra's may be the same or different from each other in a same ring. Rb's are each independently an alkyl group, an alkyloxy group or an alkylthio group, which has 1 to 10 carbon atoms, an aryl group, an aryloxy group or an arylthio group, which has 6 to 10 carbon atoms, a cycloalkyl group having 3 to 10 carbon atoms, a halogen atom, a hydroxyl group, or a mercapto group, and r represents an integer value of 0 to 3. When r is 2 to 3, Rb's may be the same or different from each other in a same ring. n is an average number of repeating units, and represents a numerical value of 0.95 to 10.0. When the r and the q are 0, Ra and Rb each refer to a hydrogen atom.

Since the maleimide has an indane scaffold, the proportion of polar functional groups in the structure of the maleimide is smaller than that of the conventional maleimide. Therefore, a cured product produced using the maleimide is preferable because it has excellent dielectric properties. Further, a cured product using a conventional maleimide resin tends to be brittle and may be inferior in brittleness resistance. However, since the maleimide has an indane scaffold, it is preferable because it has excellent elastic behavior and flexibility, and is expected to have improved brittleness resistance.

Further, it is preferable that Ra of the general formula (1) is any one of an alkyl group having 1 to 4 carbon atoms, a cycloalkyl group having 3 to 6 carbon atoms, and an aryl group having 6 to 10 carbon atoms. By using the aforementioned alkyl group having 1 to 4 carbon atoms or the like, it becomes a preferable embodiment in which the solvent solubility is improved due to the decrease in flatness and crystallinity in the vicinity of the maleimide group, and a cured product can be obtained without impairing the reactivity of the maleimide group.

The q in the general formula (1) is preferably 2 to 3, and more preferably 2. When the q is 2, the influence of steric hindrance is small, the electron density on an aromatic ring is improved, and this is a preferable embodiment in the production (synthesis) of maleimide.

In the general formula (1), it is preferable that r is 0 and Rb is a hydrogen atom. Further, it is preferable that r is 1 to 3 and Rb is at least one selected from the group consisting of an alkyl group having 1 to 4 carbon atoms, a cycloalkyl group having 3 to 6 carbon atoms, and an aryl group having 6 to 10 carbon atoms. In particular, when the r is 0 and Rb is a hydrogen atom, steric hindrance is reduced during the formation of the indane scaffold in maleimide. This is advantageous for the production (synthesis) of maleimide, and is a preferable embodiment.

<Production Method of Maleimide Having an Indane Scaffold>

A method for producing the maleimide will be described below.

In the following general formula (2), Rc's each independently represent a monovalent functional group selected from the group consisting of the following general formulae (3) and (4), the ortho position of at least one of two Rc's is a hydrogen atom, and Rb and r are compounds representing the same as above.

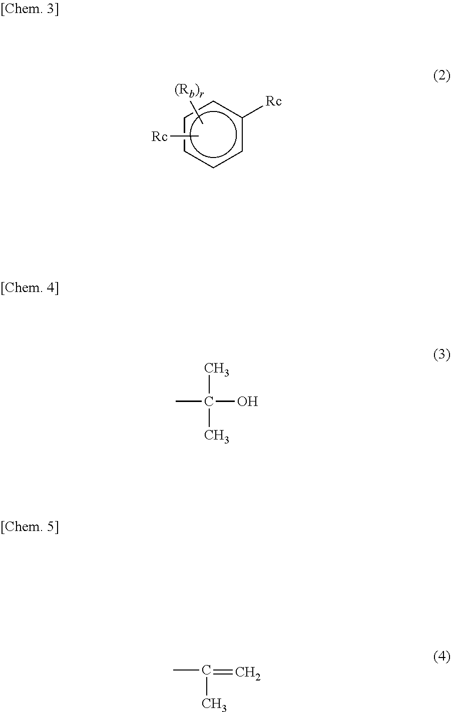

In the following general formula (5), at least one of the ortho position and para position of an amino group is a hydrogen atom, and Ra and q are each an aniline representing the same as above, or a derivative thereof. By reacting the compound of the general formula (2) and the compound of the following general formula (5) in the presence of an acid catalyst, an intermediate amine compound represented by the following general formula (6) can be obtained. Ra, Rb, q, and r in the following general formula (6) represent the same as above.

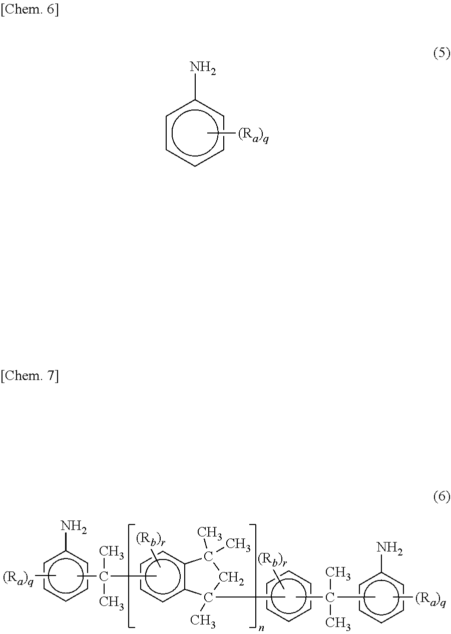

The intermediate amine compound represented by the general formula (6) contains, in its structure, the following general formula (7) having an indane scaffold. However, it becomes the structure represented by the following general formula (8) when, in the aniline represented by the general formula (5) or a derivative thereof, q is 3 or less and at least two of the ortho and para positions of the amino group are hydrogen atoms. In the following general formula (8), Ra, Rb, q, and r represent the same as above, and m is the number of repeating units and represents an integer value of 1 to 20. In addition, the structure represented by the following general formula (8) may also be included in the structure of the general formula (6).

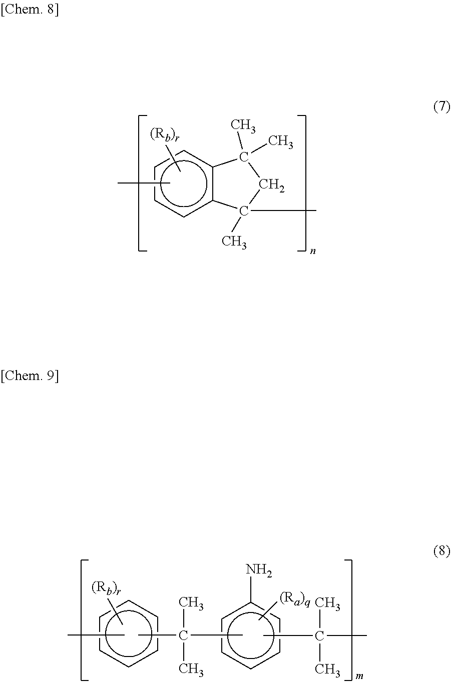

In the indane scaffold (see the general formula (7)), which is a feature of the maleimide, n, which is the average number of repeating units, is 0.95 to 10.0, preferably 0.98 to 8.0, more preferably 1.0 to 7.0, and further more preferably 1.1 to 6.0 as the average number of repeating units (average value) n in order to have a low melting point (low softening point), low melt viscosity, and excellent handleability. Since the maleimide has an indane scaffold in its structure, it is excellent in solvent solubility as compared with maleimides used so far, and is a preferable embodiment. When the n is less than 0.95, the maleimide has a high content ratio of high-melting-point substances in the structure and has poor solvent solubility. Moreover, since the proportion of high molecular weight components contributing to elastic behavior is low, the brittleness resistance of the obtained cured product is decreased, and the elastic behavior and flexibility may also be decreased, which is not preferable. Further, when the n is greater than 10.0, the viscosity becomes high when dissolved in a solvent, and the obtained cured product may have inferior heat resistance. Moreover, there are too many high molecular weight components, and when molding the cured product, the fluidity may decrease and the handleability may be inferior, which is not preferable. In addition, from the viewpoint of high thermal deformation temperature, high glass transition temperature, etc. of the cured product, the value of n is particularly preferably 0.98 to 8.0.

The compound represented by the general formula (2) used in the invention (hereinafter referred to as “compound (a)”) is not particularly limited; however, it is typically p- and m-diisopropenylbenzene, p- and m-bis(α-hydroxyisopropyl) benzene, 1-(α-hydroxyisopropyl)-3-isopropenylbenzene, 1-(α-hydroxyisopropyl)-4-isopropenylbenzene, or a mixture thereof. Further, nucleus alkyl group substituents of these compounds such as diisopropenyltoluene and bis(α-hydroxyisopropyl)toluene may also be used. Moreover, nucleus halogen substituents such as chlorodiisopropenylbenzene and chlorobis(α-hydroxyisopropyl) benzene may also be used.

In addition, examples of the compound (a) include 2-chloro-1,4-diisopropenylbenzene, 2-chloro-1,4-bis(α-hydroxyisopropyl) benzene, 2-bromo-1,4-diisopropenylbenzene, 2-bromo-1,4-bis(α-hydroxyisopropyl) benzene, 2-bromo-1,3-diisopropenylbenzene, 2-bromo-1,3-bis(α-hydroxyisopropyl) benzene, 4-bromo-1,3-diisopropylbenzene, 4-bromo-1,3-bis(α-hydroxyisopropyl) benzene, 5-bromo-1,3-diisopropenylbenzene, 5-bromo-1,3-bis(α-hydroxyisopropyl) benzene, 2-methoxy-1,4-diisopropenylbenzene, 2-methoxy-1,4-bis(α-hydroxyisopropyl) benzene, 5-ethoxy-1,3-diisopropenylbenzene, 5-ethoxy-1,3-bis(α-hydroxyisopropyl) benzene, 2-phenoxy-1,4-diisopropenylbenzene, 2-phenoxy-1,4-bis(α-hydroxyisopropyl) benzene, 2,4-diisopropenylbenzenethiol, 2,4-bis(α-hydroxyisopropyl) benzenethiol, 2,5-diisopropenylbenzenethiol, 2,5-bis(α-hydroxyisopropyl) benzenethiol, 2-methylthio-1,4-diisopropenylbenzene, 2-metylthio-1,4-bis(α-hydroxyisopropyl) benzene, 2-phenylthio-1,3-diisopropenylbenzene, 2-phenylthio-1,3-bis(α-hydroxyisopropyl) benzene, 2-phenyl-1,4-diisopropenylbenzene, 2-phenyl-1,4-bis(α-hydroxyisopropyl) benzene, 2-cyclopentyl-1,4-diisopropenylbenzene, 2-cyclopentyl-1,4-bis(α-hydroxyisopropyl) benzene, 5-naphthyl-1,3-diisopropenylbenzene, 5-naphthyl-1,3-bis(α-hydroxyisopropyl) benzene, 2-methyl-1,4-diisopropenylbenzene, 2-methyl-1,4-bis(α-hydroxyisopropyl) benzene, 5-butyl-1,3-diisopropylbenzene, 5-butyl-1,3-bis(α-hydroxyisopropyl) benzene, 5-cyclohexyl-1,3-diisopropenylbenzene, and 5-cyclohexyl-1,3-bis(α-hydroxyisopropyl) benzene.

The substituent contained in the compound (a) is not particularly limited, and the above exemplified compounds can be used. However, in the case of a substituent having a large steric hindrance, stacking between the obtained maleimides and crystallization between the maleimides are less likely to occur as compared with a substituent having a small steric hindrance. That is, the solvent solubility of the maleimide is improved, which is a preferable embodiment.

Further, for the compound represented by the general formula (5) (hereinafter referred to as “compound (b)”), in addition to aniline which is typical, dimethylaniline, diethylaniline, diisopropylaniline, ethylmethylaniline, cyclobutylaniline, cyclopentylaniline, cyclohexylaniline, chloroaniline, dichloroaniline, toluidine, xylidine, phenylaniline, nitroaniline, aminophenol, cyclohexylaniline and the like may be used. Moreover, examples thereof also include methoxyaniline, ethoxyaniline, phenoxyaniline, naphthoxyaniline, aminothiol, methylthioaniline, ethylthioaniline and phenylthioaniline.

When the maleimide group is directed bonded to the benzene ring as in the conventional maleimide (for example, N-phenylmaleimide), the benzene ring and the 5-membered ring of the maleimide are aligned on the same plane and this state is stable. Therefore, stacking becomes easy and high crystallinity is exhibited, resulting in poor solvent solubility. In contrast, in the case of the invention, the compound (b) is not particularly limited, and in addition to the above exemplified compounds that can be used, for example, when a methyl group is contained as a substituent, like 2,6-dimethylaniline, the benzene ring and the 5-membered ring of the maleimide have a twisted conformation due to the steric hindrance of the methyl group. This makes stacking difficult, and thus the crystallinity is lowered and the solvent solubility is improved, which is a preferable embodiment. However, when the steric hindrance is too large, there is a concern that the reactivity during synthesis of the maleimide may be hindered. Therefore, for example, it is preferable to use the compound (b) having an alkyl group having 2 to 4 carbon atoms.

In the method for producing an intermediate amine compound represented by the general formula (6) used in the invention, a maleimide having an indane scaffold can be obtained by feeding and reacting the compound (a) and the compound (b) where a molar ration of the compound (b) to the compound (a) (compound (b)/compound (a)) is preferably 0.1 to 2.0, more preferably 0.2 to 1.0 (first step), and further adding the compound (b) in an amount of preferably 0.5 to 20.0, more preferably 0.7 to 5.0 in terms of molar ratio to the previously added compound (a) and reacting the mixture (second step). Further, this two-step reaction gives favorable results in order to complete the reaction or from the viewpoint of handleability and the like. In the first-step reaction, the compound (b) is preferably added in an amount of, by molar ratio to the previously added compound (a) (compound (b)/compound (a)), 0.10 to 0.49, more preferably 0.20 to 0.39. As a result, with a wide molecular weight distribution, the content ratio of high-melting-point substances having a low molecular weight is low, and the proportion of high molecular weight components is high. Therefore, it is preferable because an intermediate amine compound having excellent solvent solubility and further contributing to elastic behavior and brittleness resistance, and a maleimide can be obtained.

Examples of an acid catalyst used in the reaction include inorganic acids such as phosphoric acid, hydrochloric acid and sulfuric acid, organic acids such as oxalic acid, benzensulfonic acid, toluenesulfonic acid, methanesulfonic acid and fluoromethanesulfonic acid, solid acids such as activated clay, acid clay, silica alumina, zeolite and strongly acidic ion exchange resins, and heteropolyhydrochloric acids. However, a solid acid that can be easily removed as a catalyst by filtration after the reaction is preferable from the viewpoint of handleability, and when other acids are used, it is preferable to neutralize with a base and wash with water after the reaction.

The acid catalyst is blended in an amount of 5 to 40 parts by mass with respect to a total amount of 100 parts by mass of the initially fed raw materials, i.e., the compound (a) and the compound (b). However, from the viewpoint of handleability and economy, 5 to 30 parts by mass is preferable. The reaction temperature is usually only necessary in the range of 100 to 300° C. However, in order to suppress the formation of isomer structures and avoid side reactions such as thermal decomposition, it is preferably 150 to 230° C.

As for the reaction time, the reaction does not proceed completely in a short time, and side reactions such as a thermal decomposition reaction of the product occur when the reaction time is long. Therefore, under the aforementioned reaction temperature conditions, it is usually in the range of 2 to 24 hours in total, preferably in the range of 4 to 12 hours in total. In order to reduce the low molecular weight components and increase the high molecular weight components, it is more preferably in the range of 8 to 12 hours.

In the method for producing the intermediate amine compound, aniline or a derivative thereof also serves as a solvent, and thus it is not necessary to use another solvent. However, it is also possible to use a solvent. For example, in the case of a reaction system that also serves as a dehydration reaction, specifically, in the case of reaction with a compound having an α-hydroxypropyl group is used as a raw material, it is possible to use a method in which the dehydration reaction is completed using a solvent capable of azeotropic dehydration, such as toluene, xylene, and chlorobenzene, and then the solvent is distilled off and the reaction is carried out within the above reaction temperature range.

The maleimide used in the invention may be obtained by feeding the intermediate amine compound represented by the general formula (6) obtained by the above method into a reactor and dissolving in an appropriate solvent, then reacting in the presence of maleic anhydride and a catalyst, and after the reaction, removing unreacted maleic anhydride and other impurities by washing with water or the like, and removing the solvent by reducing pressure. Moreover, a dehydrating agent may also be used at the time of reaction.

The maleimide used in the invention has the scaffold of the general formula (1) and includes the structure represented by the general formula (7) having the indane scaffold. However, when q is 3 or less and at least two of the ortho and para positions of the amino group are hydrogen atoms, the structure corresponding to the general formula (8), that is, a structure represented by the following general formula (9), is included as a structure represented by the general formula (1).

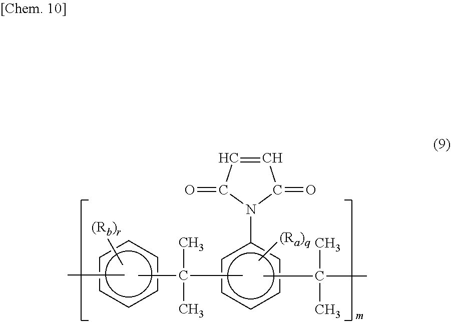

Ra, Rb, q, r and m in the general formula (9) indicate the same as above.

Examples of the organic solvent used in the maleimization reaction for synthesizing the maleimide include ketones such as acetone, methyl ethyl ketone (MEK), methyl isobutyl ketone, cyclohexanone and acetophenone, aprotic solvents such as N,N-dimethylformamide, N,N-dimethylacetamide, dimethyl sulfoxide, N-methyl-2-pyrrolidone, acetonitrile and sulfolane, cyclic ethers such as dioxane and tetrahydrofuran, esters such as ethyl acetate and butyl acetate, and aromatic solvents such as benzene, toluene and xylene. These may be used alone or in combination.

It is a preferable embodiment that in the maleimization reaction, the intermediate amine compound and the maleic anhydride are blended and fed with the equivalent ratio of the maleic anhydride to the amino equivalent of the intermediate amine compound of preferably 1 to 1.5, more preferably 1.1 to 1.2, and reacted in an organic solvent having a mass ratio of 0.5 to 50, preferably 1 to 5 with respect to the total amount of the intermediate amine compound and the maleic anhydride.

Examples of the catalyst used in the maleimidization reaction include acetates of nickel, cobalt, sodium, calcium, iron, lithium, manganese, etc, inorganic salts such as chlorides, bromides, sulfates and nitrates, inorganic acids such as phosphoric acid, hydrochloric acid and sulfuric acid, organic acids such as oxalic acid, benzenesulfonic acid, toluenesulfonic acid, methanesulfonic acid and fluoromethanesulfonic acid, solid acids such as activated clay, acid clay, silica-alumina, zeolite and strongly acidic ion exchange resins, and heteropolyhydrochloric acid, and in particularly, toluenesulfonic acid is preferably used.

Examples of the dehydrating agent used in the maleimidization reaction include lower aliphatic carboxylic acid anhydrides such as acetic anhydride, propionic anhydride and butyric anhydride, oxides such as phosphorus pentoxide, calcium oxide and barium oxide, inorganic acids such as sulfuric acid, and porous ceramics such as molecular sieves, and acetic anhydride may be preferably used.

The amount of the catalyst and dehydrating agent used in the maleimidization reaction is not particularly limited. However, usually, based on 1 equivalent of the amino group of the intermediate amine compound, the catalyst may be used in an amount of 0.0001 to 1.0 mol, preferably 0.01 to 0.3 mol, and the dehydrating agent may be used in an amount of 1 to 3 mol, preferably 1 to 1.5 mol.

As reaction conditions for the maleimidization reaction, the intermediate amine compound and the maleic anhydride may be fed and reacted in a temperature range of 10 to 100° C., preferably 30 to 50° C., for 0.5 to 12 hours, preferably 1 to 4 hours, and then the catalyst may be added to react in a temperature range of 90 to 130° C., preferably 105 to 120° C., for 2 to 24 hours, preferably 4 to 10 hours, more preferably 6 to 10 hours for the reduction of the low molecular weight components and the increase of the high molecular weight components. Further, after the reaction, removing unreacted maleic anhydride and other impurities by washing with water or the like and heat aging also reduces the low molecular weight components and increase the high molecular weight components.

From the viewpoint of excellent low dielectric constant and low dielectric loss tangent, the maleimide preferably has a molecular weight distribution (weight average molecular weight (Mw)/number average molecular weight (Mn)) calculated from gel permeation chromatography (GPC) measurement of 1.0 to 4.0, more preferably 1.1 to 3.8, further more preferably 1.2 to 3.6, and particularly preferably 1.3 to 3.4. From the GPC chart obtained from the GPC measurement, when the molecular weight distribution is wide and there are many high molecular weight components, the proportion of the high molecular weight components that contribute to elastic behavior is high. Therefore, compared with a cured product using the conventional maleimide, brittleness is suppressed, and a cured product excellent in elastic behavior and flexibility can be obtained, which is a preferable embodiment.

<Gpc Measurement>

Based on gel permeation chromatography (GPC) under the following conditions, the molecular weight distribution of maleimide (weight average molecular weight (Mw)/number average molecular weight (Mn)) and the average number “n” of repeating units that contribute to the indane scaffold in the maleimide were measured and calculated based on the number average molecular weight (Mn).

Measuring device: “HLC-8320 GPC” manufactured by Tosoh Corporation

Column: “HXL-L” manufactured by Tosoh Corporation+“TSK-GEL G2000HXL” manufactured by Tosoh Corporation+“TSK-GEL G2000HXL” manufactured by Tosoh Corporation+“TSK-GEL G3000HXL” manufactured by Tosoh Corporation+“TSK-GEL G4000HXL” manufactured by Tosoh Corporation

Detector: RI (differential refractometer)

Data processing: “GPC workstation EcoSEC-WorkStation” manufactured by Tosoh Corporation

Measuring conditions: Column temperature 40° C.
Developing solvent tetrahydrofuran
Flow rate 1.0 ml/min

Standard: The following monodisperse polystyrenes having a known molecular weight were used in accordance with the measurement manual of the aforementioned “GPC workstation EcoSEC-WorkStation”.

(Polystyrene Used)
“A-500” manufactured by Tosoh Corporation
“A-1000” manufactured by Tosoh Corporation
“A-2500” manufactured by Tosoh Corporation
“A-5000” manufactured by Tosoh Corporation
“F-1” manufactured by Tosoh Corporation
“F-2” manufactured by Tosoh Corporation
“F-4” manufactured by Tosoh Corporation
“F-10” manufactured by Tosoh Corporation
“F-20” manufactured by Tosoh Corporation
“F-40” manufactured by Tosoh Corporation
“F-80” manufactured by Tosoh Corporation
“F-128” manufactured by Tosoh Corporation

Sample: 1.0% by mass of tetrahydrofuran solution in terms of resin solid content of maleimide obtained in the synthesis example filtered with a microfilter (50 μl).

The maleimide of the invention preferably contains 32 area % or less, more preferably 30 area % or less, further preferably 28 area % or less, of the maleimide having an average number n of repeating units of 0 in 100 area % of the total amount of the maleimide based on the GPC measurement. As the content ratio (area %) of the maleimide in which the n is 0 is small, the content ratio of the low molecular weight components having high crystallinity is reduced, the solubility in the solvent is improved, and the dissolved state can be maintained for a long period of time, which is a preferable embodiment. In order to reduce the content ratio of the maleimide in which the n is 0, it is possible to prepare by reducing the molar ratio between the compound (a) and the compound (b) (compound (b)/compound (a)) in the process of producing the intermediate amine compound. Further, it can also be appropriately prepared depending on the amount of catalyst, the reaction temperature, and the reaction time in the process of producing the intermediate amine compound.

### <Preparation of Curable Resin Composition>

The curable resin composition of the invention preferably contains the maleimide. Since the maleimide is excellent in solvent solubility, fluidity during heat melting, and handleability, and further can contribute to brittleness resistance, heat resistance, low dielectric constant and low dielectric loss tangent, a cured product obtained by the curable resin composition containing the maleimide is excellent in brittleness resistance, elastic behavior, flexibility, heat resistance, and dielectric properties. Therefore, it is a preferable embodiment.

In the curable resin composition of the invention, a curing agent, and if necessary, various formulations such as a curing accelerator, a silane coupling agent, a mold releasing agent, a pigment, an emulsifier, a non-halogen flame retardant, and an inorganic filler may be added. Further, in addition to the maleimide, an epoxy resin, a phenol resin, an active ester resin, a cyanate resin, etc. may be appropriately blended as long as the object of the invention is not impaired.

### <Cured Product>

The cured product of the invention is preferably obtained by the curable agent composition. The cured product can be obtained by subjecting the curable resin composition to a curing reaction. The curable resin composition is obtained by uniformly mixing each of the aforementioned components, and can be easily made into a cured product by a method which is the same as a conventionally known method. Examples of the cured product include molded cured products such as laminates, cast products, adhesive layers, coating films, and films.

### <Heat-Resistant Materials and Electronic Materials>

Since the cured product obtained by the curable resin composition containing the maleimide of the invention is excellent in heat resistance and dielectric properties, it can be suitably used for heat-resistant members and electronic members. In particular, the cured product can be suitably used for prepregs, circuit boards, semiconductor sealing materials, semiconductor devices, build-up films, build-up boards, adhesives using conductive pastes, resist materials, etc. Further, it can also be suitably used for a matrix resin of a fiber reinforced resin, and is particularly suitable as a prepreg having high heat resistance. In addition, the maleimide having the indane scaffold contained in the curable resin composition can be made into paint because it exhibits excellent solubility in various solvents. Heat-resistant members and electronic members thus obtained can be suitably used for various purposes such as industrial machine parts, general machine parts, parts for automobile, railroad, vehicles and the like, space and/or aviation related parts, electronic and/or electrical parts, building materials, container and/or packaging members, daily necessities, sports and/or leisure goods, and wind power generation housing members. However, they are not limited thereto.

### EXAMPLES

Next, the invention will be specifically described with reference to Examples and Comparative Examples. In the following description, “part” and “%” are based on mass unless otherwise specified. The softening point, amine equivalent, GPC, and FD-MS spectrum were measured and evaluated under the following conditions.

1) Softening Point

Measuring method: The softening point (° C.) of the intermediate amine compound obtained in the synthetic example shown below was measured in accordance with JIS K7234 (ring and ball method).

2) Amine Equivalent

The amine equivalent of the intermediate amine compound was measured by the following measuring method.

Approximately 2.5 g of the sample intermediate amine compound, 7.5 g of pyridine, 2.5 g of anhydrous acetic acid, and 7.5 g of triphenylphosphine were precisely weighed in a 500 mL Erlenmeyer flask with a stopper, and then a cooling tube was attached thereto and the mixture was heated to reflux in an oil bath set at 120° C. for 150 minutes.

After cooling, 5.0 mL of distilled water, 100 mL of propylene glycol monomethyl ether, and 75 mL of tetrahydrofuran were added, and titration was performed with a 0.5 mol/L potassium hydroxide-ethanol solution by a potentiometric titration method. A blank test was performed in the same manner to make corrections.

Amine equivalent (g/eq.)=(S×2,000)/(Blank−A)

S: Amount of sample (g)

A: Consumption (mL) of 0.5 mol/L of potassium hydroxide-ethanol solution

Blank: Consumption (mL) of 0.5 mol/L of potassium hydroxide-ethanol solution in the blank test

3) GPC Measurement

Measurement was performed using the following measuring device and measuring conditions, and GPC charts (FIGS. 1 to 10) of maleimide obtained in the synthetic example shown below were obtained. From the results of the GPC charts, the molecular weight distribution (weight average molecular weight (Mw)/number average molecular weight (Mn)) and the average number “n” of repeating units that contribute to the indane scaffold in the maleimide were measured and calculated based on the number average molecular weight (Mn). Specifically, for compounds in which n is 0 to 4, theoretical molecular weights and respective measured molecular weights in GPC were plotted on a scatter plot, and an approximate straight line was drawn. The number average molecular weight (Mn) was obtained from points indicated by measured values Mn (1) on the straight line, and n was calculated. Further, the content ratio (area %) of the maleimide having an average number n of repeating units of in 100 are % of a total amount of the maleimide was calculated based on the GPC measurement.

Measuring device: “HLC-8320 GPC” manufactured by Tosoh Corporation

Column: “HXL-L” manufactured by Tosoh Corporation+“TSK-GEL G2000HXL” manufactured by Tosoh Corporation+“TSK-GEL G2000HXL” manufactured by Tosoh Corporation+“TSK-GEL G3000HXL” manufactured by Tosoh Corporation+“TSK-GEL G4000HXL” manufactured by Tosoh Corporation

Detector: RI (differential refractometer)

Data processing: “GPC workstation EcoSEC-WorkStation” manufactured by Tosoh Corporation

Measuring conditions: Column temperature 40° C.
Developing solvent tetrahydrofuran
Flow rate 1.0 ml/min

Standard: The following monodisperse polystyrenes having a known molecular weight were used in accordance with the measurement manual of the aforementioned “GPC workstation EcoSEC-WorkStation”.

(Polystyrene Used)
“A-500” manufactured by Tosoh Corporation
“A-1000” manufactured by Tosoh Corporation
“A-2500” manufactured by Tosoh Corporation
“A-5000” manufactured by Tosoh Corporation
“F-1” manufactured by Tosoh Corporation
“F-2” manufactured by Tosoh Corporation
“F-4” manufactured by Tosoh Corporation
“F-10” manufactured by Tosoh Corporation
“F-20” manufactured by Tosoh Corporation
“F-40” manufactured by Tosoh Corporation
“F-80” manufactured by Tosoh Corporation
“F-128” manufactured by Tosoh Corporation

Sample: 1.0% by mass of tetrahydrofuran solution in terms of resin solid content of maleimide obtained in the synthesis example filtered with a microfilter (50 μl).

4) FD-MS Spectrum

The FD-MS spectrum was measured using the following measuring device and measuring conditions.

Measuring device: JMS-T100GC AccuTOF

Measuring condition

Measuring range: m/z=4.00 to 2000.00

Rate of change: 51.2 mA/min

Final current value: 45 mA

Cathode voltage: −10 kV

Recording interval: 0.07 sec

### [Synthesis Example 1] Synthesis of Maleimide Compound A-1

(1) Synthesis of Intermediate Amine Compound

In a 1 L flask equipped with a thermometer, a cooling tube, a Dean Stark trap, and a stirrer, 48.5 g (0.4 mol) of 2,6-dimethylaniline, 272.0 g (1.4 mol) of α,α′-dihydroxy-1,3-diisopropylbenzene, 280 g of xylene and 70 g of activated clay were fed and were heated to 120° C. with stirring. Further, distillate was removed with a Dean Stark tube while raising the temperature to 210° C., and the mixture was allowed to react for 3 hours. Then, the mixture was cooled to 140° C., and after 145.4 g (1.2 mol) of 2,6-dimethylaniline was fed, the temperature was raised to 220° C. and the mixture was allowed to react for 3 hours. After the reaction, the mixture was air-cooled to 100° C., diluted with 300 g of toluene, the activated clay was removed by filtration and low molecular weight substances such as solvents and unreacted substances were distilled off under reduced pressure. In this way, 364.1 g of an intermediate amine compound represented by the following general formula (A-1) was obtained. The amine equivalent was 298 and the softening point was 70° C.

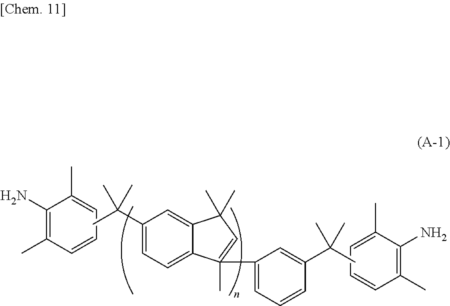

(2) Maleimidization

In a 2 L flask equipped with a thermometer, a cooling tube, a Dean Stark trap, and a stirrer, 131.8 g (1.3 mol) of maleic anhydride and 700 g of toluene were fed and were stirred at room temperature. Next, a mixed solution of 364.1 g of the reaction product (A-1) and 175 g of DMF was added dropwise over 1 hour.

After completion of the dropping, the mixture was allowed to react at room temperature for another 2 hours. 37.1 g of p-toluenesulfonic acid monohydrate was added, and the reaction solution was heated while azeotropic water and toluene under reflux were cooled and separated, and thereafter, only toluene was returned to the system and a dehydration reaction was carried out for 8 hours. After air cooling to room temperature, the mixture was subjected to vacuum concentration and a brown solution was dissolved in 600 g of ethyl acetate, washed 3 times with 150 g of ion-exchanged water and 3 times with 150 g of a 2% sodium hydrogen carbonate aqueous solution. After adding sodium sulfate and drying, the reaction product obtained by vacuum concentration was vacuum dried at 80° C. for 4 hours, and 413.0 g of a product containing the maleimide compound A-1 was obtained. In the FD-MS spectrum of the maleimide compound A-1, peaks of M+=560, 718, and 876 were found, and the peaks correspond to the cases where n is 0, 1, and 2, respectively. When obtaining the value of the number of repeating units n (based on the number average molecular weight) in the indane scaffold in the maleimide A-1 by GPC, the GPC chart was as shown in FIG. 1, where n was 1.47 and the molecular weight distribution (Mw/Mn) was 1.81. In addition, the maleimide having an average number of repeating units n of 0 was 26.5 area % in 100 area % of the total amount of the maleimide A-1.

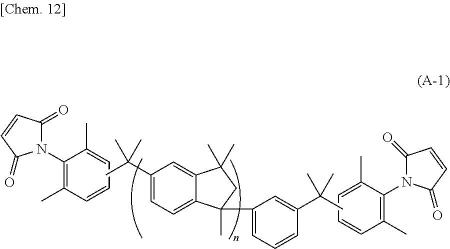

### [Synthesis Example 2] Synthesis of Maleimide Compound A-2

(1) Synthesis of Intermediate Amine Compound

In a 1 L flask equipped with a thermometer, a cooling tube, a Dean Stark trap, and a stirrer, 48.5 g (0.4 mol) of 2,6-dimethylaniline, 233.2 g (1.2 mol) of α,α′-dihydroxy-1,3-diisopropylbenzene, 230 g of xylene and 66 g of activated clay were fed and were heated to 120° C. with stirring. Further, distillate was removed with a Dean Stark tube while raising the temperature to 210° C., and the mixture was allowed to react for 3 hours. Then, the mixture was cooled to 140° C., and after 145.4 g (1.2 mol) of 2,6-dimethylaniline was fed, the temperature was raised to 220° C. and the mixture was allowed to react for 3 hours. After the reaction, the mixture was air-cooled to 100° C., diluted with 300 g of toluene, the activated clay was removed by filtration and low molecular weight substances such as solvents and unreacted substances were distilled off under reduced pressure. In this way, 278.4 g of an intermediate amine compound represented by the following general formula (A-2) was obtained. The amine equivalent was 294 and the softening point was 65° C.

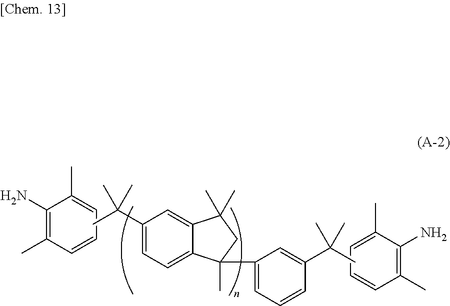

(2) Maleimidization

In a 2 L flask equipped with a thermometer, a cooling tube, a Dean Stark trap, and a stirrer, 107.9 g (1.1 mol) of maleic anhydride and 600 g of toluene were fed and were stirred at room temperature. Next, a mixed solution of 278.4 g of the reaction product (A-2) and 150 g of DMF was added dropwise over 1 hour.

After completion of the dropping, the mixture was allowed to react at room temperature for another 2 hours. 27.0 g of p-toluenesulfonic acid monohydrate was added, and the reaction solution was heated while azeotropic water and toluene under reflux were cooled and separated, and thereafter, only toluene was returned to the system and a dehydration reaction was carried out for 8 hours. After air cooling to room temperature, the mixture was subjected to vacuum concentration and a brown solution was dissolved in 500 g of ethyl acetate, washed 3 times with 120 g of ion-exchanged water and 3 times with 120 g of a 2% sodium hydrogen carbonate aqueous solution. After adding sodium sulfate and drying, the reaction product obtained by vacuum concentration was vacuum dried at 80° C. for 4 hours, and 336.8 g of a product containing the maleimide compound A-2 was obtained. In the FD-MS spectrum of the maleimide compound A-2, peaks of M+=560, 718, and 876 were found, and the peaks correspond to the cases where n is 0, 1, and 2, respectively. When obtaining the value of the number of repeating units n (based on the number average molecular weight) in the indane scaffold in the maleimide A-2 by GPC, the GPC chart was as shown in FIG. 2, where n was 1.25 and the molecular weight distribution (Mw/Mn) was 3.29. In addition, the maleimide having an average number of repeating units n of 0 was 33.7 area % in 100 area % of the total amount of the maleimide A-2.

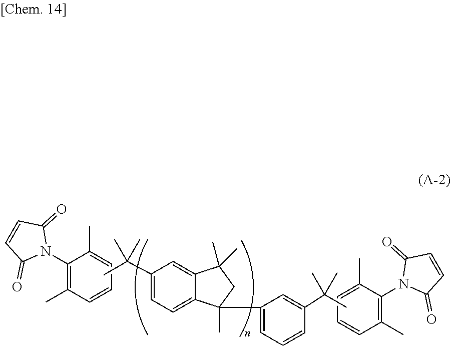

### [Synthesis Example 3] Synthesis of Maleimide Compound A-3

(1) Synthesis of Intermediate Amine Compound

In a 2 L flask equipped with a thermometer, a cooling tube, a Dean Stark trap, and a stirrer, 48.5 g (0.4 mol) of 2,6-dimethylaniline, 388.6 g (2.0 mol) of α,α′-dihydroxy-1,3-diisopropylbenzene, 350 g of xylene and 123 g of activated clay were fed and were heated to 120° C. with stirring. Further, distillate was removed with a Dean Stark tube while raising the temperature to 210° C., and the mixture was allowed to react for 3 hours. Then, the mixture was cooled to 140° C., and after 145.4 g (1.2 mol) of 2,6-dimethylaniline was fed, the temperature was raised to 220° C. and the mixture was allowed to react for 3 hours. After the reaction, the mixture was air-cooled to 100° C., diluted with 500 g of toluene, the activated clay was removed by filtration and low molecular weight substances such as solvents and unreacted substances were distilled off under reduced pressure. In this way, 402.1 g of an intermediate amine compound represented by the following general formula (A-3) was obtained. The amine equivalent was 306 and the softening point was 65° C.

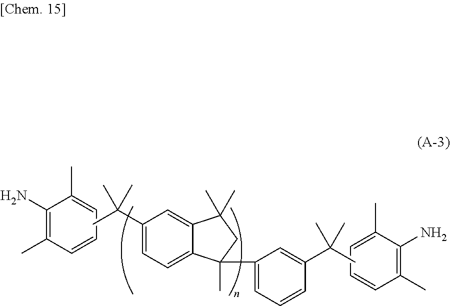

(2) Maleimidization

In a 2 L flask equipped with a thermometer, a cooling tube, a Dean Stark trap, and a stirrer, 152.1 g (1.5 mol) of maleic anhydride and 700 g of toluene were fed and were stirred at room temperature. Next, a mixed solution of 402.1 g of the reaction product (A-3) and 200 g of DMF was added dropwise over 1 hour.

After completion of the dropping, the mixture was allowed to react at room temperature for another 2 hours. 37.5 g of p-toluenesulfonic acid monohydrate was added, and the reaction solution was heated while azeotropic water and toluene under reflux were cooled and separated, and thereafter, only toluene was returned to the system and a dehydration reaction was carried out for 8 hours. After air cooling to room temperature, the mixture was subjected to vacuum concentration and a brown solution was dissolved in 800 g of ethyl acetate, washed 3 times with 200 g of ion-exchanged water and 3 times with 200 g of a 2% sodium hydrogen carbonate aqueous solution. After adding sodium sulfate and drying, the reaction product obtained by vacuum concentration was vacuum dried at 80° C. for 4 hours, and 486.9 g of a product containing the maleimide compound A-3 was obtained. In the FD-MS spectrum of the maleimide compound A-3, peaks of M+=560, 718, and 876 were found, and the peaks correspond to the cases where n is 0, 1, and 2, respectively. When obtaining the value of the number of repeating units n (based on the number average molecular weight) in the indane scaffold in the maleimide A-3 by GPC, the GPC chart was as shown in FIG. 3, where n was 1.96 and the molecular weight distribution (Mw/Mn) was 1.52. In addition, the maleimide having an average number of repeating units n of 0 was 17.1 area % in 100 area % of the total amount of the maleimide A-3.

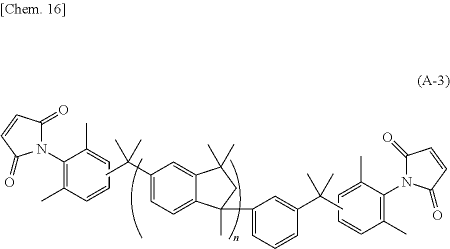

### [Synthesis Example 4] Synthesis of Maleimide Compound A-4

(1) Synthesis of Intermediate Amine Compound

In a 2 L flask equipped with a thermometer, a cooling tube, a Dean Stark trap, and a stirrer, 59.7 g (0.4 mol) of 2,6-diethylaniline, 272.0 g (1.4 mol) of α,α′-dihydroxy-1,3-diisopropylbenzene, 350 g of xylene and 94 g of activated clay were fed and were heated to 120° C. with stirring. Further, distillate was removed with a Dean Stark tube while raising the temperature to 210° C., and the mixture was allowed to react for 3 hours. Then, the mixture was cooled to 140° C., and after 179.1 g (1.2 mol) of 2,6-diethylaniline was fed, the temperature was raised to 220° C. and the mixture was allowed to react for 3 hours. After the reaction, the mixture was air-cooled to 100° C., diluted with 500 g of toluene, the activated clay was removed by filtration and low molecular weight substances such as solvents and unreacted substances were distilled off under reduced pressure. In this way, 342.1 g of an intermediate amine compound represented by the following general formula (A-4) was obtained. The amine equivalent was 364 and the softening point was 47° C.

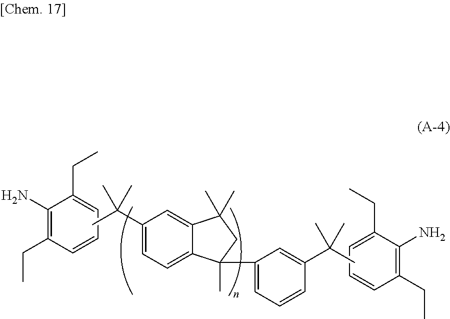

(2) Maleimidization

In a 2 L flask equipped with a thermometer, a cooling tube, a Dean Stark trap, and a stirrer, 107.9 g (1.1 mol) of maleic anhydride and 600 g of toluene were fed and were stirred at room temperature. Next, a mixed solution of 342.1 g of the reaction product (A-4) and 180 g of DMF was added dropwise over 1 hour.

After completion of the dropping, the mixture was allowed to react at room temperature for another 2 hours. 26.8 g of p-toluenesulfonic acid monohydrate was added, and the reaction solution was heated while azeotropic water and toluene under reflux were cooled and separated, and thereafter, only toluene was returned to the system and a dehydration reaction was carried out for 8 hours. After air cooling to room temperature, the mixture was subjected to vacuum concentration and a brown solution was dissolved in 500 g of ethyl acetate, washed 3 times with 200 g of ion-exchanged water and 3 times with 200 g of a 2% sodium hydrogen carbonate aqueous solution. After adding sodium sulfate and drying, the reaction product obtained by vacuum concentration was vacuum dried at 80° C. for 4 hours, and 388.1 g of a product containing the maleimide compound A-4 was obtained. In the FD-MS spectrum of the maleimide compound A-4, peaks of M+=616, 774, and 932 were found, and the peaks correspond to the cases where n is 0, 1, and 2, respectively. When obtaining the value of the number of repeating units n (based on the number average molecular weight) in the indane scaffold in the maleimide A-4 by GPC, the GPC chart was as shown in FIG. 4, where n was 1.64 and the molecular weight distribution (Mw/Mn) was 1.40. In addition, the maleimide having an average number of repeating units n of 0 was 15.8 area % in 100 area % of the total amount of the maleimide A-4.

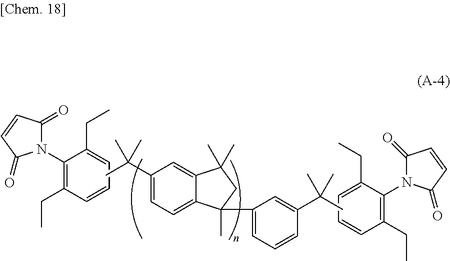

### [Synthesis Example 5] Synthesis of Maleimide Compound A-5

(1) Synthesis of Intermediate Amine Compound

In a 1 L flask equipped with a thermometer, a cooling tube, a Dean Stark trap, and a stirrer, 70.9 g (0.4 mol) of 2,6-diisopropylaniline, 272.0 g (1.4 mol) of α,α′-dihydroxy-1,3-diisopropylbenzene, 350 g of xylene and 97 g of activated clay were fed and were heated to 120° C. with stirring. Further, distillate was removed with a Dean Stark tube while raising the temperature to 210° C., and the mixture was allowed to react for 3 hours. Then, the mixture was cooled to 140° C., and after 212.7 g (1.2 mol) of 2,6-diisopropylaniline was fed, the temperature was raised to 220° C. and the mixture was allowed to react for 3 hours. After the reaction, the mixture was air-cooled to 100° C., diluted with 500 g of toluene, the activated clay was removed by filtration and low molecular weight substances such as solvents and unreacted substances were distilled off under reduced pressure. In this way, 317.5 g of an intermediate amine compound represented by the following general formula (A-5) was obtained. The amine equivalent was 366 and the softening point was 55° C.

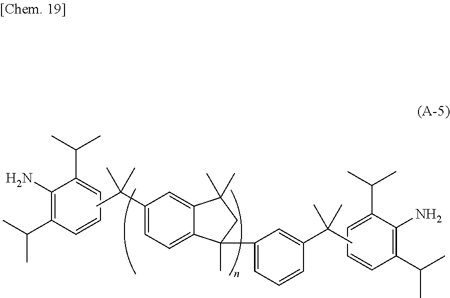

(2) Maleimidization

In a 2 L flask equipped with a thermometer, a cooling tube, a Dean Stark trap, and a stirrer, 107.9 g (1.1 mol) of maleic anhydride and 600 g of toluene were fed and were stirred at room temperature. Next, a mixed solution of 317.5 g of the reaction product (A-5) and 175 g of DMF was added dropwise over 1 hour.

After completion of the dropping, the mixture was allowed to react at room temperature for another 2 hours. 24.8 g of p-toluenesulfonic acid monohydrate was added, and the reaction solution was heated while azeotropic water and toluene under reflux were cooled and separated, and thereafter, only toluene was returned to the system and a dehydration reaction was carried out for 8 hours. After air cooling to room temperature, the mixture was subjected to vacuum concentration and a brown solution was dissolved in 600 g of ethyl acetate, washed 3 times with 200 g of ion-exchanged water and 3 times with 200 g of a 2% sodium hydrogen carbonate aqueous solution. After adding sodium sulfate and drying, the reaction product obtained by vacuum concentration was vacuum dried at 80° C. for 4 hours, and 355.9 g of a product containing the maleimide compound A-5 was obtained. In the FD-MS spectrum of the maleimide compound A-5, peaks of M+=672, 830, and 988 were found, and the peaks correspond to the cases where n is 0, 1, and 2, respectively. When obtaining the value of the number of repeating units n (based on the number average molecular weight) in the indane scaffold in the maleimide A-5 by GPC, the GPC chart was as shown in FIG. 5, where n was 1.56 and the molecular weight distribution (Mw/Mn) was 1.24. In addition, the maleimide having an average number of repeating units n of 0 was 20.2 area % in 100 area % of the total amount of the maleimide A-5.

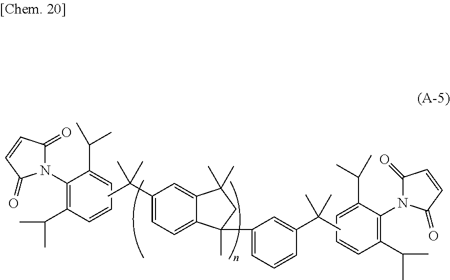

### [Synthesis Example 6] Synthesis of Maleimide Compound A-7

(1) Synthesis of Intermediate Amine Compound

In a 1 L flask equipped with a thermometer, a cooling tube, a Dean Stark trap, and a stirrer, 48.5 g (0.4 mol) of 2,6-dimethylaniline, 194.3 g (1.0 mol) of α,α′-dihydroxy-1,3-diisopropylbenzene, 204 g of xylene and 53 g of activated clay were fed and were heated to 120° C. with stirring. Further, distillate was removed with a Dean Stark tube while raising the temperature to 210° C., and the mixture was allowed to react for 3 hours. Then, the mixture was cooled to 140° C., and after 168.4 g (1.4 mol) of 2,6-dimethylaniline was fed, the temperature was raised to 220° C. and the mixture was allowed to react for 3 hours. After the reaction, the mixture was air-cooled to 100° C., diluted with 300 g of toluene, the activated clay was removed by filtration and low molecular weight substances such as solvents and unreacted substances were distilled off under reduced pressure. In this way, 256.4 g of an intermediate amine compound represented by the following general formula (A-7) was obtained. The amine equivalent was 292 and the softening point was 64° C.

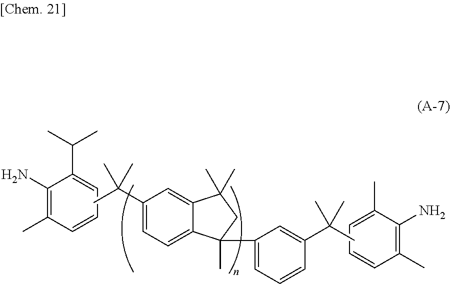

(2) Maleimidization

In a 2 L flask equipped with a thermometer, a cooling tube, a Dean Stark trap, and a stirrer, 107.9 g (1.1 mol) of maleic anhydride and 600 g of toluene were fed and were stirred at room temperature. Next, a mixed solution of 256.4 g of the reaction product (A-7) and 150 g of DMF was added dropwise over 1 hour.

After completion of the dropping, the mixture was allowed to react at room temperature for another 2 hours. 28.5 g of p-toluenesulfonic acid monohydrate was added, and the reaction solution was heated while azeotropic water and toluene under reflux were cooled and separated, and thereafter, only toluene was returned to the system and a dehydration reaction was carried out for 8 hours. After air cooling to room temperature, the mixture was subjected to vacuum concentration and a brown solution was dissolved in 500 g of ethyl acetate, washed 3 times with 120 g of ion-exchanged water and 3 times with 120 g of a 2% sodium hydrogen carbonate aqueous solution. After adding sodium sulfate and drying, the reaction product obtained by vacuum concentration was vacuum dried at 80° C. for 4 hours, and 319.6 g of a product containing the maleimide compound A-1 was obtained. In the FD-MS spectrum of the maleimide compound A-7, peaks of M+=560, 718, and 876 were found, and the peaks correspond to the cases where n is 0, 1, and 2, respectively. When obtaining the value of the number of repeating units n (based on the number average molecular weight) in the indane scaffold in the maleimide A-7 by GPC, the GPC chart was as shown in FIG. 6, where n was 0.92 and the molecular weight distribution (Mw/Mn) was 1.45. In addition, the maleimide having an average number of repeating units n of 0 was 38.8 area % in 100 area % of the total amount of the maleimide A-7.

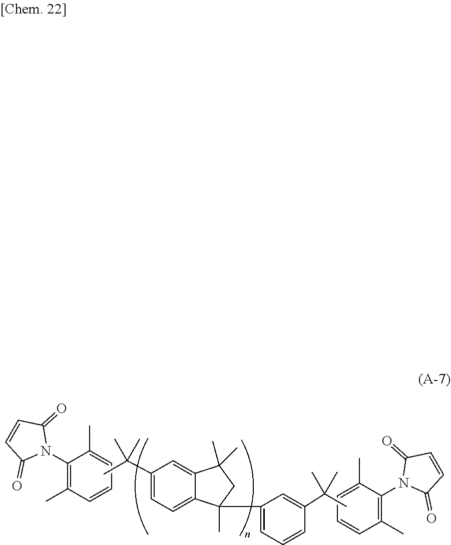

### [Synthesis Example 7] Synthesis of Maleimide Compound A-8

(1) Synthesis of Intermediate Amine Compound

The same operation as in the method for synthesizing the intermediate amine compound A-1 was carried out except that the reaction time at 210° C. was changed to 6 hours and the reaction time at 220° C. was changed to 3 hours, and 345.2 g of an intermediate amine compound represented by the following general formula (A-8) was obtained. The amine equivalent was 348 and the softening point was 71° C.

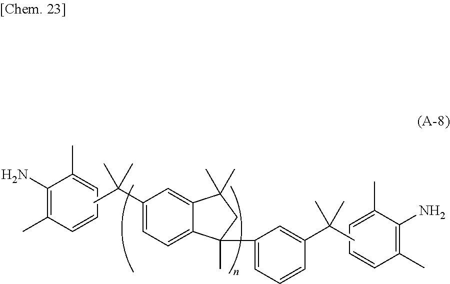

(2) Maleimidization

407.6 g of a product containing the maleimide compound A-8 was obtained by operating the same as in the method for synthesizing the maleimide compound A-1 except that the intermediate was replaced with A-8. In the FD-MS spectrum of the maleimide compound A-8, peaks of M+=560, 718, and 876 were found, and the peaks correspond to the cases where n is 0, 1, and 2, respectively. When obtaining the value of the number of repeating units n (based on the number average molecular weight) in the indane scaffold in the maleimide A-8 by GPC, the GPC chart was as shown in FIG. 7, where n was 2.59 and the molecular weight distribution (Mw/Mn) was 1.49. In addition, the maleimide having an average number of repeating units n of 0 was 14.9 area % in 100 area % of the total amount of the maleimide A-8.

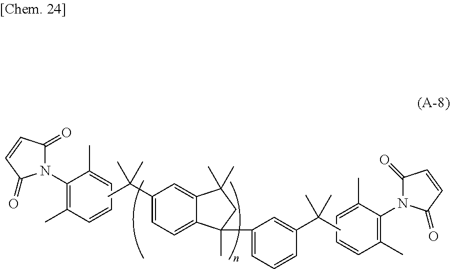

### [Synthesis Example 8] Synthesis of Maleimide Compound A-9

The same operation as in the method for synthesizing the intermediate amine compound A-1 was carried out by changing the reaction time at 210° C. to 6 hours and the reaction time at 220° C. to 3 hours, and by subjecting to the same conditions as in the method for synthesizing the maleimide compound A-1 except that the dehydration reaction under reflux in the maleimidization reaction was set to 10 hours for the synthesized intermediate amine compound (amine equivalent: 347, softening point: 71° C.), 415.6 g of a product containing the maleimide compound A-9 was obtained. In the FD-MS spectrum of the maleimide compound A-9, peaks of M+=560, 718, and 876 were found, and the peaks correspond to the cases where n is 0, 1, and 2, respectively. When obtaining the value of the number of repeating units n (based on the number average molecular weight) in the indane scaffold in the maleimide A-9 by GPC, the GPC chart was as shown in FIG. 8, where n was 2.91 and the molecular weight distribution (Mw/Mn) was 1.64. In addition, the maleimide having an average number of repeating units n of 0 was 14.2 area % in 100 area % of the total amount of the maleimide A-9.

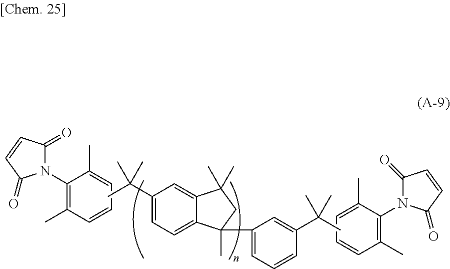

### [Synthesis Example 9] Synthesis of Maleimide Compound A-10

The same operation as in the method for synthesizing the intermediate amine compound A-1 was carried out by changing the reaction time at 210° C. to 9 hours and the reaction time at 220° C. to 3 hours, and by subjecting to the same conditions as in the method for synthesizing the maleimide compound A-1 except that the dehydration reaction under reflux in the maleimidization reaction was set to 10 hours for the synthesized intermediate amine compound (amine equivalent: 342, softening point: 69° C.), 398.7 g of a product containing the maleimide compound A-10 was obtained. In the FD-MS spectrum of the maleimide compound A-10, peaks of M+=560, 718, and 876 were found, and the peaks correspond to the cases where n is 0, 1, and 2, respectively. When obtaining the value of the number of repeating units n (based on the number average molecular weight) in the indane scaffold in the maleimide A-10 by GPC, the GPC chart was as shown in FIG. 9, where n was 3.68 and the molecular weight distribution (Mw/Mn) was 2.09. In addition, the maleimide having an average number of repeating units n of 0 was 12.4 area % in 100 area % of the total amount of the maleimide A-10.

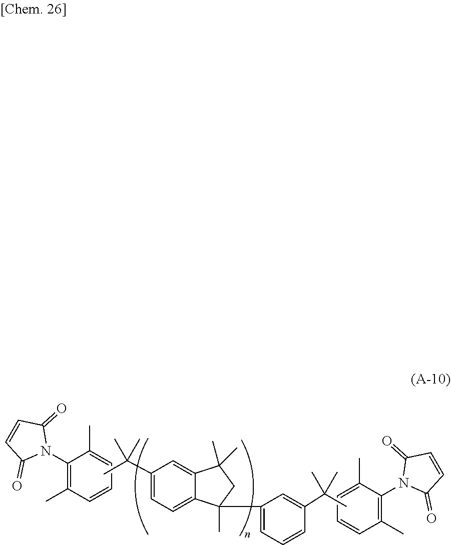

### [Synthesis Example 10] Synthesis of Maleimide Compound A-11

The same operation as in the method for synthesizing the intermediate amine compound A-1 was carried out by changing the reaction time at 210° C. to 9 hours and the reaction time at 220° C. to 3 hours, and by subjecting to the same conditions as in the method for synthesizing the maleimide compound A-1 except that the dehydration reaction under reflux in the maleimidization reaction was set to 12 hours for the synthesized intermediate amine compound (amine equivalent: 347, softening point: 70° C.), 422.7 g of a product containing the maleimide compound A-11 was obtained. In the FD-MS spectrum of the maleimide compound A-11, peaks of M+=560, 718, and 876 were found, and the peaks correspond to the cases where n is 0, 1, and 2, respectively. When obtaining the value of the number of repeating units n (based on the number average molecular weight) in the indane scaffold in the maleimide A-11 by GPC, the GPC chart was as shown in FIG. 10, where n was 4.29 and the molecular weight distribution (Mw/Mn) was 3.02. In addition, the maleimide having an average number of repeating units n of 0 was 11.0 area % in 100 area % of the total amount of the maleimide A-11.

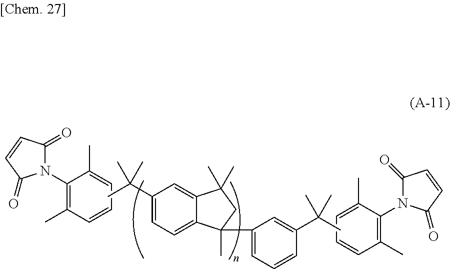

### Examples 1 to 9, and Comparative Examples 1 to 2

<Solvent Solubility of Maleimide>

The solubility of the maleimides (A-1) to (A-5) and (A-7) to (A-11) obtained in Synthesis Examples 1 to 10 and a commercially available maleimide (A-6) (4,4,′-diphenylmethanebismaleimide, “BMI-1000” manufactured by Daiwa Kasei Industry Co., Ltd.) for comparison in toluene and methyl ethyl ketone (MEK) was evaluated, and the evaluation results are shown in Table 1.

As a method for evaluating solvent solubility, toluene solutions and methyl ethyl ketone (MEK) solutions were prepared by using the maleimides obtained in the above Synthesis Examples and the Comparative Examples such that a non-volatile content is 10, 20, 30, 40, 50, 60, and 70% by mass, respectively.

Specifically, vials containing the maleimides obtained in the above Synthesis Examples and the Comparative Examples respectively were left at room temperature (25° C.) for 60 days, and in each solution of each non-volatile composition, the case where it was uniformly dissolved (without insoluble matter) was (visually) evaluated as A, and the case where it was not dissolved (with insoluble matter) was (visually) evaluated as B. When the non-volatile content is 20% by mass or more, if it can be dissolved in a solvent, it is practically preferable.

TABLE 1

| Evaluation of solvent solubility | | Ex. 1 | Ex. 2 | Ex. 3 | Ex. 4 | Ex. 5 | Ex. 6 | Ex. 7 | Ex. 8 | Ex. 9 | C.E. 1 | C.E. 2 |
|---|---|---|---|---|---|---|---|---|---|---|---|---|
| Maleimide | | A-1 | A-2 | A-3 | A-4 | A-5 | A-8 | A-9 | A-10 | A-11 | A-6 | A-7 |
| Non-volatile content | 10% | A | A | A | A | A | A | A | A | A | B | A |
| in toluene solution | 20% | A | A | A | A | A | A | A | A | A | B | B |
| | 30% | A | B | A | A | A | A | A | A | A | B | B |
| | 40% | B | B | A | A | A | A | A | A | A | B | B |
| | 50% | B | B | A | A | A | A | A | A | A | B | B |
| | 60% | B | B | A | B | A | A | A | A | A | B | B |
| | 70% | B | B | A | B | B | A | A | A | A | B | B |
| Non-volatile content | 10% | A | A | A | A | A | A | A | A | A | B | A |
| in MEK solution | 20% | A | A | A | A | A | A | A | A | A | B | A |
| | 30% | A | A | A | A | A | A | A | A | A | B | A |
| | 40% | A | A | A | A | A | A | A | A | A | B | B |
| | 50% | A | A | A | A | A | A | A | A | A | B | B |
| | 60% | B | B | A | A | A | A | A | A | A | B | B |
| | 70% | B | B | A | A | A | A | A | A | A | B | B |

From the evaluation results in Table 1 above, it has been found that in Examples 1 to 9, since maleimides having an indane scaffold were used, when preparing the toluene solution, they could be dissolved even if the non-volatile content was 20% by mass, and when preparing the MEK solution, they could be dissolved even if the non-volatile content was 50% by mass, which have excellent solvent solubility. On the other hand, it has been found that the commercially available maleimide used in Comparative Example 1 does not have an indane scaffold in its structure and is inferior in solvent solubility. In addition, in Comparative Example 2, it has been found that although a maleimide having an indane scaffold was used, the solvent solubility was poor because the average number n of repeating units of the indane scaffold portion was not included in the desired range.

### INDUSTRIAL APPLICABILITY

Since the cured product obtained by using the maleimide is excellent in heat resistance and dielectric properties, the maleimide of the invention can be suitably used for heat-resistant members and electronic members and in particular, can be suitably used for semiconductor sealing materials, circuit boards, build-up films, build-up boards, adhesives and resist materials. Further, it can also be suitably used for a matrix resin of a fiber reinforced resin, and is particularly suitable as a prepreg having high heat resistance.

### BRIEF DESCRIPTION OF DRAWINGS

FIG. 1 is a GPC chart of the maleimide compound (A-1) obtained in Synthesis Example 1.

FIG. 2 is a GPC chart of the maleimide compound (A-2) obtained in Synthesis Example 2.

FIG. 3 is a GPC chart of the maleimide compound (A-3) obtained in Synthesis Example 3.

FIG. 4 is a GPC chart of the maleimide compound (A-4) obtained in Synthesis Example 4.

FIG. 5 is a GPC chart of the maleimide compound (A-5) obtained in Synthesis Example 5.

FIG. 6 is a GPC chart of the maleimide compound (A-7) obtained in Synthesis Example 6.

FIG. 7 is a GPC chart of the maleimide compound (A-8) obtained in Synthesis Example 7.

FIG. 8 is a GPC chart of the maleimide compound (A-9) obtained in Synthesis Example 8.

FIG. 9 is a GPC chart of the maleimide compound (A-10) obtained in Synthesis Example 9.

FIG. 10 is a GPC chart of the maleimide compound (A-11) obtained in Synthesis Example 10.

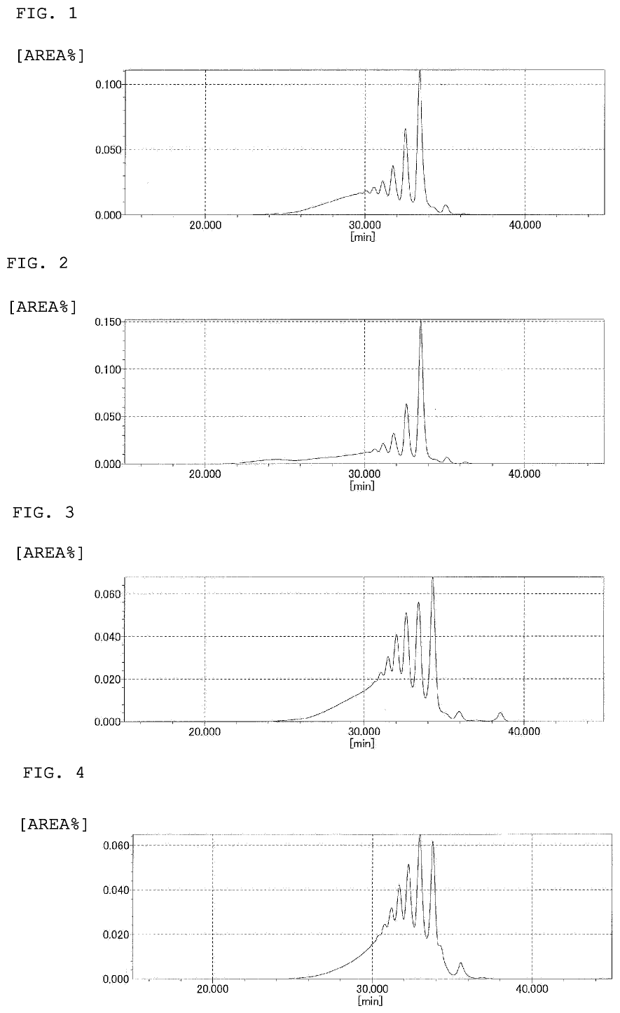

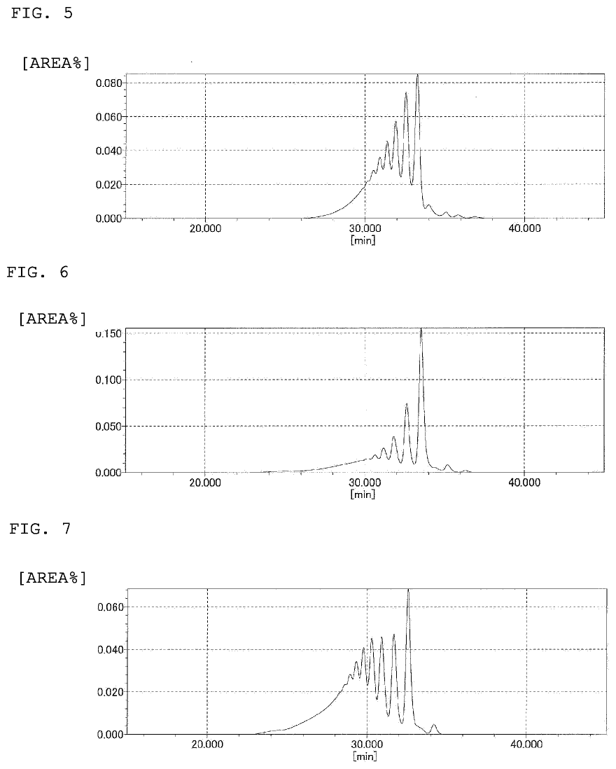

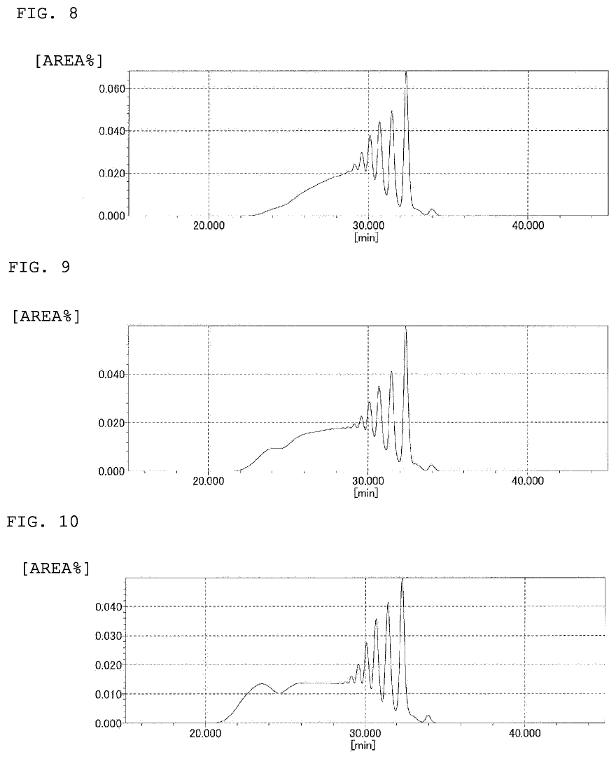

1. A maleimide having an indane scaffold represented by the following general formula (1): 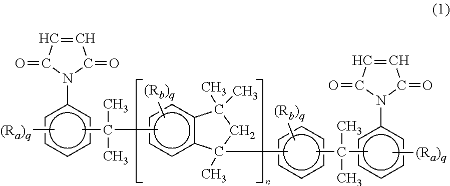
in formula (1), Ra's are each independently an alkyl group, an alkyloxy group or an alkylthio group, which has 1 to 10 carbon atoms, an aryl group, an aryloxy group or an arylthio group, which has 6 to 10 carbon atoms, a cycloalkyl group having 3 to 10 carbon atoms, a halogen atom, a nitro group, a hydroxyl group, or a mercapto group, and q represents an integer value of 0 to 4; when q is 2 to 4, Ra's may be the same or different from each other in a same ring; Rb's are each independently an alkyl group, an alkyloxy group or an alkylthio group, which has 1 to 10 carbon atoms, an aryl group, an aryloxy group or an arylthio group, which has 6 to 10 carbon atoms, or a cycloalkyl group having 3 to 10 carbon atoms, a halogen atom, a hydroxyl group, or a mercapto group, and r represents an integer value of 0 to 3; when r is 2 to 3, Rb's may be the same or different from each other in a same ring; and n is an average number of repeating units, and represents a numerical value of 0.95 to 10.0.

2. The maleimide according to claim 1, wherein a molecular weight distribution (Mw/Mn) obtained by GPC measurement is 1.0 to 4.0.

3. The maleimide according to claim 1, wherein Ra in the formula (1) is at least one selected from the group consisting of an alkyl group having 1 to 4 carbon atoms, a cycloalkyl group having 3 to 6 carbon atoms, and an aryl group having 6 to 10 carbon atoms.

4. The maleimide according to claim 1, wherein q in the formula (1) is 2 to 3.

5. The maleimide according to claim 1, wherein r in the formula (1) is 0 and Rb is a hydrogen atom.

6. The maleimide according to claim 1, wherein r in the formula (1) is 1 to 3, and Rb is at least one selected from the group consisting of an alkyl group having 1 to 4 carbon atoms, a cycloalkyl group having 3 to 6 carbon atoms, and an aryl group having 6 to 10 carbon atoms.

7. The maleimide according to claim 1, wherein in 100% by mass of a total amount of the maleimide, the maleimide in which n is 0 is contained in 32 area % or less.

8. A curable resin composition containing the maleimide according to claim 1.

9. A cured product obtained by the curable resin composition according to claim 8.

10. The maleimide according to claim 2, wherein Ra in the formula (1) is at least one selected from the group consisting of an alkyl group having 1 to 4 carbon atoms, a cycloalkyl group having 3 to 6 carbon atoms, and an aryl group having 6 to 10 carbon atoms.

11. The maleimide according to claim 2, wherein q in the formula (1) is 2 to 3.

12. The maleimide according to claim 3, wherein q in the formula (1) is 2 to 3.

13. The maleimide according to claim 2, wherein r in the formula (1) is 0 and Rb is a hydrogen atom.

14. The maleimide according to claim 3, wherein r in the formula (1) is 0 and Rb is a hydrogen atom.

15. The maleimide according to claim 2, wherein r in the formula (1) is 1 to 3, and Rb is at least one selected from the group consisting of an alkyl group having 1 to 4 carbon atoms, a cycloalkyl group having 3 to 6 carbon atoms, and an aryl group having 6 to 10 carbon atoms.

16. The maleimide according to claim 3, wherein r in the formula (1) is 1 to 3, and Rb is at least one selected from the group consisting of an alkyl group having 1 to 4 carbon atoms, a cycloalkyl group having 3 to 6 carbon atoms, and an aryl group having 6 to 10 carbon atoms.

17. The maleimide according to claim 2, wherein in 100% by mass of a total amount of the maleimide, the maleimide in which n is 0 is contained in 32 area % or less.

18. The maleimide according to claim 3, wherein in 100% by mass of a total amount of the maleimide, the maleimide in which n is 0 is contained in 32 area % or less.

19. A curable resin composition containing the maleimide according to claim 2.

20. A curable resin composition containing the maleimide according to claim 3.
